(19) United States
(12) Patent Application Publication
(10) Pub. No.: US 2011/0282322 A1
(43) Pub. Date: Nov. 17, 2011

(54) Hemodialysis Access Needle Assembly System and Method

(76) Inventors: John K. Edoga, North Beach, NJ (US); Thierry Richard, Villiers Le Muhieu (FR)

(21) Appl. No.: 13/125,313

(22) PCT Filed: Oct. 27, 2009
(86) PCT No.: PCT/US09/62144

§ 371 Date: Jun. 8, 2011

Related U.S. Application Data
(62) Division of application No. 61/108,690, filed on Oct. 27, 2008.

(51) Int. Cl. A61M 5/46

(52) U.S. Cl. 604/506; 604/117

(57) ABSTRACT

An access needle assembly for use with implanted hemodialysis access ports is disclosed. In some embodiments, the assembly includes the following: a tubular needle having an internal cavity including an open leading end having a beveled edge and an open trailing end opposite the leading end, the cavity including a side outflow opening adjacent the trailing end; an obturator configured to be removably positioned within the tubular needle; an interface portion including a central cavity so that the interface portion is positioned adjacent the opening, the interface including a flanged portion extending substantially perpendicular from the central cavity and positioned concentrically over the opening; a conduit portion fluidly connected with the cavity via the flanged portion; and a depth block joined with the interface portion below the flanged portion, the depth block including a lower end opposite the interface portion.

### CROSS REFERENCE TO PRIOR APPLICATION

This application claims the benefit of U.S. Provisional Application No. 61/108,690, filed Oct. 27, 2008 which is incorporated by reference as if disclosed herein in its entirety.

### BACKGROUND

The disclosed subject matter generally relates to a needle assembly, systems, and methods used in hemodialysis treatments. More particularly, the disclosed subject matter relates to hemodialysis access needles, systems, and methods for use with an implanted access port to facilitate positioning of a needle end in an artery of a patient.

The present invention relates to access needles used with access ports implantable into a mammal to gain access to veins and arteries thereof. Typically, such ports are implanted for use during hemodialysis. The access ports of the present invention may be utilized as an alternative to a typical arteriovenous (hereinafter “AV”) fistula, AV graft, or large central venous catheter used during modern kidney dialysis procedures. These ports are designed to raise the comfort level of a dialysis patient and to reduce the risk of access damage while also reducing the effort of the medical staff required to conduct the dialysis.

Unfortunately, a significant number of individuals suffer from decreased kidney function. If the kidney function is depreciated enough, usually to approximately 10% of normal levels, an individual must either undergo kidney dialysis procedures or receive a kidney transplant. Dialysis procedures remove toxic substances, waste, and bodily fluids from the bloodstream when the kidneys are unable to do so. Presently, two types of dialysis are commonly utilized, peritoneal dialysis and hemodialysis.

Peritoneal dialysis generally involves injecting special solutions into the abdomen of a patient through a port, or plastic tube. The special solution enters the abdomen and occupies the space around the abdominal organs known as the peritoneal cavity. Wastes, toxins, and excess bodily fluids mix with the special solution and are retained therein through osmosis. Once the special solution absorbs a sufficient amount of the wastes, toxins, and excess fluids, the combination may be drained out through the port. This process can either occur every four to six hours in a manual procedure, or continuously if used in conjunction with a cycler machine. While this procedure may usually be performed at home by the patient it will be appreciated that such a process creates a great burden on the patient, and typically interferes with normal life functioning.

Hemodialysis is conducted by circulating blood through an external filtering machine. Typically, a patient will require hemodialysis three-times per week, with each session lasting approximately four hours.

In hemodialysis, an “arterial” catheter removes blood from the body. The blood is then pumped across a semi-permeable membrane containing solutions to remove toxins, wastes, and excess bodily fluids. The cleansed blood is then returned to the body through a “venous” catheter. Other than in emergency situations, dialysis access is generally obtained through an AV fistula or AV graft. The same graft serves to both supply blood to the hemodialysis machine as well as return blood to the body. In this regard, two catheters are typically placed into the AV fistula or AV graft. The catheter closest to the heart typically serves as the “arterial” catheter, flowing blood from the body, and the downstream catheter typically serves as the “venous” catheter, returning blood to the body. Because the pressure gradient between the two needles is typically not great, the hemodialysis machine must include a pump to circulate the blood.

Because, peripheral veins are typically too small in diameter to permit the required flow of 250 milliliters of blood per minute back into the body, AV fistulas are surgically created approximately six weeks before hemodialysis begins in order to artificially enlarge a vein. This is done by joining a vein to an artery in a localized area while the patient is under anesthesia. The increased blood from the artery causes the vein to enlarge and thicken, thus permitting larger flows through the vein then would otherwise be possible. After the six weeks that the fistula needs to heal, two dialysis needles may be placed within the enlarged and thickened vein. One needle permits blood to be removed for dialysis and the other permits cleansed blood to return to the enlarged and thickened vein.

For individuals whose veins are not suitable for an AV fistula, an AV graft may be used. This procedure involves surgically grafting a portion of the patient's saphenous vein, a donor animal artery, or a synthetic conduit and using it to connect an artery to an existing vein. The grafted vein or prosthetic conduit may be double punctured to draw blood into the dialysis machine and return cleansed blood into the body.

Neither AV fistulas nor AV grafts are ideal. The resulting increased blood through the veins may cause a neo-intimal hyperplasia which could occlude the veins and lead to access loss. Additionally, the direct flow of blood from an artery into the veins puts undue strain on the local vascular system in general, and the heart in particular. Finally, because blood is both withdrawn from and returned to the body in the same AV fistula, dialysis is typically inefficient because of the phenomenon of recirculation.

Recent dialysis advances involve the implanting of dialysis access ports beneath the skin. These ports generally contain a chamber plugged with a self-sealing material, such as rubberized silicone, with a synthetic catheter extending out from within the chamber. The port is placed under the skin and the catheter is surgically implanted into a vein. A second port is similarly implanted beneath the skin and its catheter is surgically implanted into another portion of the vein. One port may then be used to remove blood for dialysis while the other port is used to return the cleansed blood back to the body.

Unfortunately, even with the use of AV fistulas, AV grafts, and implanted access ports, positioning of the end of the access needle into the artery of the patient can be problematic. As a result, punctures to the artery walls and to portions of the fistulas, grafts, and access ports can occur and result in damage to the structure, which can require additional procedures to replace the damaged structure.

### SUMMARY

An access needle assembly for use with implanted hemodialysis access ports is disclosed. In some embodiments, the assembly includes the following: a tubular needle having an internal cavity including an open leading end having a beveled edge and an open trailing end opposite the leading end, the trailing end including a removable cap for closing the trailing end, the cavity including a side outflow opening adjacent the trailing end; an obturator configured to be removably positioned within the tubular needle, the obturator having a cross-section that is substantially equivalent to a cross-section of the tubular needle thereby filling the cavity when positioned within the tubular needle; an interface portion including a central cavity, the tubular needle extending through the central cavity so that the interface portion is positioned adjacent the opening, the interface including a flanged portion extending substantially perpendicular from the central cavity and positioned concentrically over the opening; a conduit portion fluidly connected with the cavity via the flanged portion of the interface portion; and a depth block joined with the interface portion below the flanged portion, the depth block including a lower end opposite the interface portion that contacts a patient thereby defining a depth that the tubular needle penetrates the patient.

A system for providing hemodialysis is disclosed. In some embodiments, the system includes the following: an access module including a hemodialysis access port for implanting in a patient, the port including a chimney adapted to be connected to a vessel such that an interior portion of the chimney is in fluid communication with the vessel, a ring adapted to fit securely within the chimney, a collar adapted to fit securely around the chimney, a plug adapted to fit securely within the ring, and a port outer casing partially surrounding the vessel and the collar, the outer port casing including an anti-compression portion adapted to prevent compression of the catheter wherein fluid flowing within the vessel is blocked from passing through the chimney by the plug; and an access needle module including an access needle assembly for use with an implanted hemodialysis access port, the access needle assembly including a tubular needle having an internal cavity including an open leading end having a beveled edge and an open trailing end opposite the leading end, the trailing end including a removable cap for closing the trailing end, the cavity including a side outflow opening adjacent the trailing end, an obturator configured to be removably positioned within the tubular needle, the obturator having a cross-section that is substantially equivalent to a cross-section of the tubular needle thereby filling the cavity when positioned within the tubular needle, an interface portion including a central cavity, the tubular needle extending through the central cavity so that the interface portion is positioned adjacent the opening, the interface including a flanged portion extending substantially perpendicular from the central cavity and positioned concentrically over the opening, a conduit portion fluidly connected with the cavity via the flanged portion of the interface portion, and a depth block joined with the interface portion below the flanged portion, the depth block including a lower end opposite the interface portion that contacts a patient thereby defining a depth that the tubular needle penetrates the patient.

A method of limiting the depth of penetration of an access needle for use with implanted hemodialysis access ports is disclosed. In some embodiments, the method includes the following: determining a length of penetration required to ensure a leading end of the access needle is positioned within a vessel of a patient; providing a tubular needle having an internal cavity including an open leading end having a beveled edge and an open trailing end opposite the leading end, the trailing end including a removable cap for closing the trailing end, the cavity including a side outflow opening adjacent the trailing end, the tubular needle including an obturator configured to be removably positioned within the tubular needle, the obturator having a cross-section that is substantially equivalent to a cross-section of the tubular needle thereby filling the cavity when positioned within the tubular needle, the needle including an interface portion including a central cavity, the tubular needle extending through the central cavity so that the interface portion is positioned adjacent the opening, the interface including a flanged portion extending substantially perpendicular from the central cavity and positioned concentrically over the opening, the tubular needle including a conduit portion fluidly connected with the cavity via the flanged portion of the interface portion; joining a depth block with the interface portion below the flanged portion, the depth block having a predetermined length and including a lower end opposite the interface portion that contacts a patient thereby defining a depth that the tubular needle penetrates the patient, wherein the predetermined length is selected so that the leading end of the access needle is positioned within a vessel of a patient; inserting a tubular needle into the vessel of the patient via the access port by bringing the lower end of the depth block in contact with the patient; removing the obturator from the tubular needle; capping the tubular needle; and injecting or withdrawing fluids to or from the patient via the conduit portion using the tubular needle.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a side section view of a system according to some embodiments according to the disclosed subject matter;

FIGS. 2-7 are views of an access needle assembly according to some embodiments of the disclosed subject matter; and

FIG. 8 is a diagram of a method according to some embodiments of the disclosed subject matter.

### DETAILED DESCRIPTION

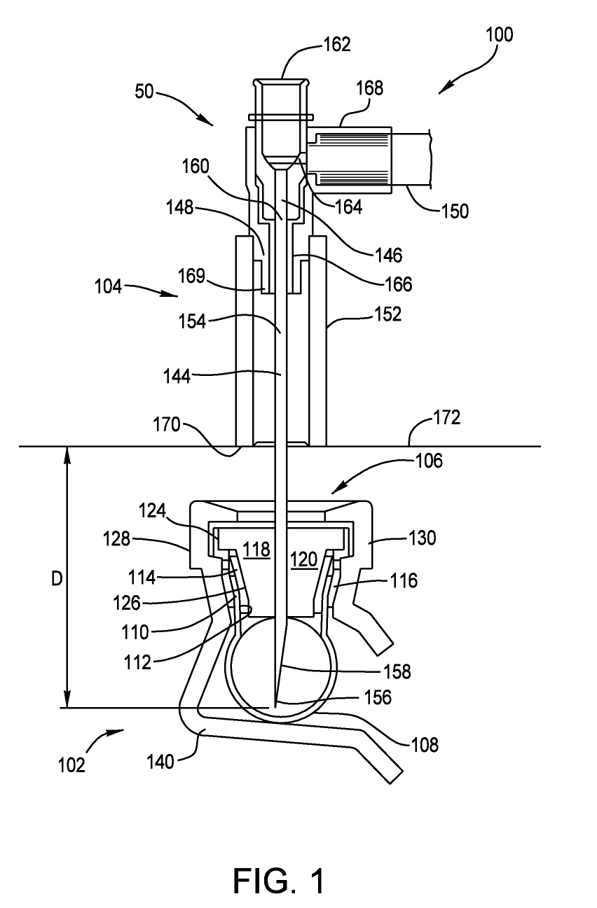

Generally, the disclosed subject matter relates to a hemodialysis access needle assembly 50 and a system 100 for providing hemodialysis, which includes hemodialysis access needle assembly 50. As shown in FIG. 1, some embodiments of system 100 include an access module 102, which is typically implanted under the skin of a patient, and an access needle module 104.

Access module 102 includes a hemodialysis access port 106 for implanting in a patient. One example of port 106 is described in published U.S. Patent Application No. 2006/0247605, filed by Edoga et al., which is hereby incorporated by reference as if disclosed herein in its entirety. Port 106 is typically mounted on a vessel, such as artery 108. Port 106 includes a sewn-on chimney 110 that is adapted to be connected to artery 108 such that an interior portion 112 of the chimney is in fluid communication with the artery. Chimney 110 is typically stitched to artery 108 in a surgical procedure. Chimney 110 is typically formed from a material such as PTFE or Dacron. Port 106 also includes a ring 114, a collar 116, and a plug 118, which are combined with chimney 110 to form a port core 120.

Ring 114 is provided to bolster chimney 110 and to shield the chimney from being compromised by a needle. Ring 114 is typically cylindrical shaped or cone shaped, and can be fitted within chimney 110. In some embodiments, ring 114 has a diameter slightly smaller than that of chimney 110 so that it fits easily inside, or equal to or just slightly larger so the chimney can be stretched over the ring. In addition, in some embodiments, the height of ring 114 matches the height or length of chimney 110.

After ring 114 is positioned within chimney 110, collar 116 is placed over the chimney. Like ring 114, collar 116 is typically cylindrical. Collar 116 typically includes a split (not shown) such that the overall diameter of the collar may be manipulated. In a natural condition, collar 116 generally has an inside diameter equal to or slightly less than that of an outside diameter of ring 114. As collar 116 is placed over chimney 110 with ring 114 already installed, the collar can be deformed into an opened state and permitted to spring back such that a tight fit around the ring and chimney is created. In some embodiments, collar 116 is not split and is pressure fitted into engagement with chimney 110 and ring 114.

Plug 118 is positioned within ring 114 to block fluids from entering or exiting through chimney 110. Plug 118 is typically a self-sealing insert but can be a mechanical device inclusive of a valve assembly. In either event, plug 118 typically includes an upper shoulder portion 124 and a lower portion 126. Lower portion 126 is typically cylindrical or cone shaped, and is generally adapted to fit tightly within ring 114 such that upper shoulder portion 124 rests above collar 116. Plug 118 is typically formed from a silicone-based material.

A pair of outer port casings 128, 130 encase port core 120. Outer port casings 128, 130 are typically held together by mechanical means, such as fasters, including bolts (not shown) and nuts (not shown). Outer port casings 128, 130 typically include an anti-compression portion 140 that is adapted to prevent compression of artery 108. As assembled, outer port casings 128, 130 are fitted around port core 120 to complete port 106.

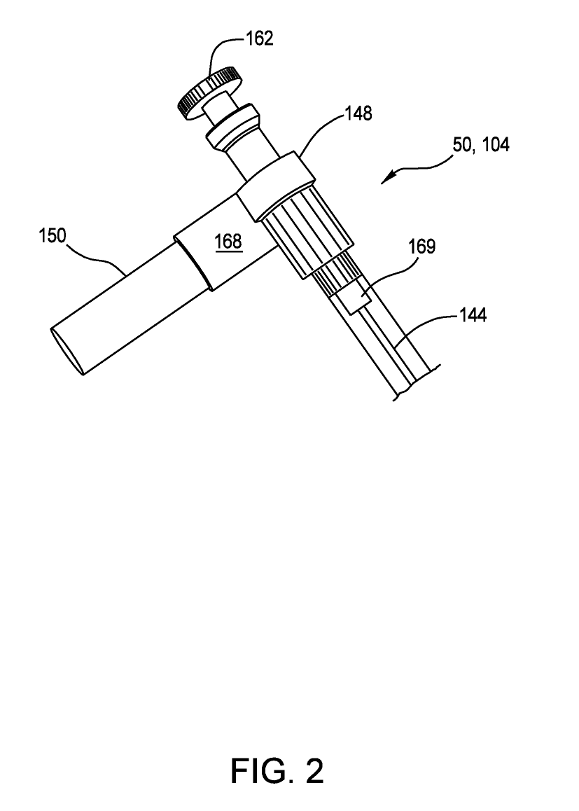

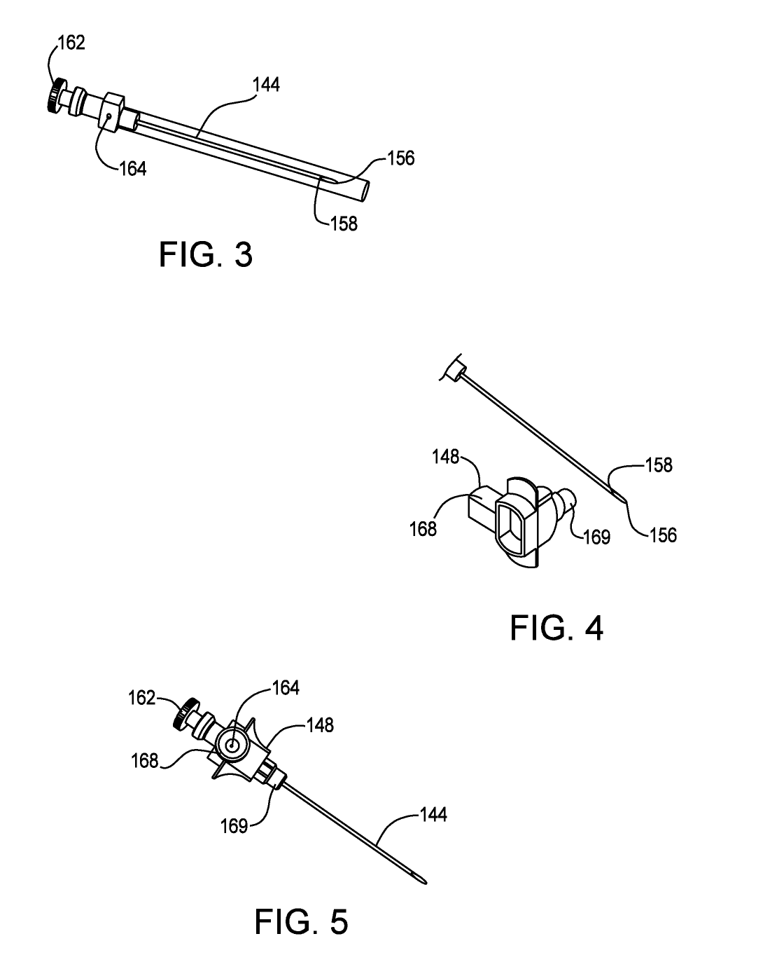

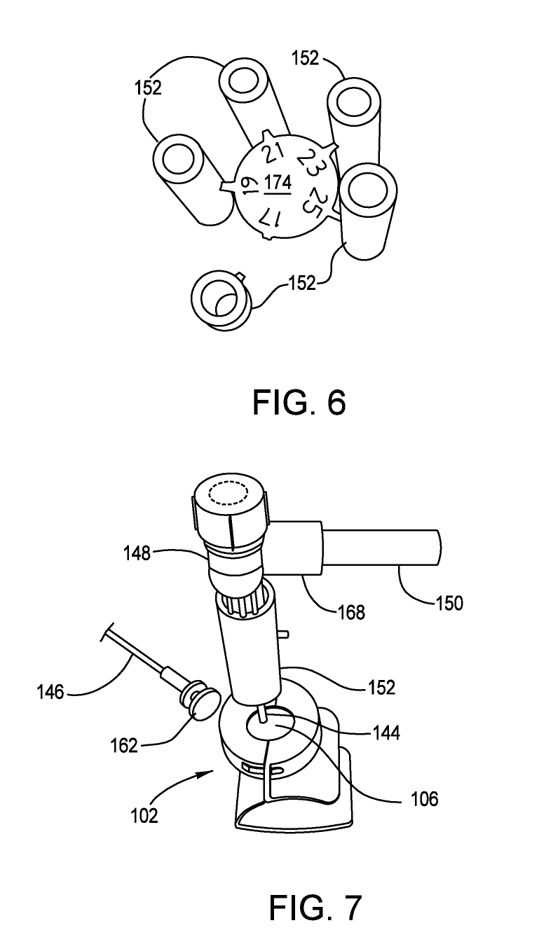

Referring now to FIGS. 1-7, access needle module 104 includes an access needle assembly 50 for use with implanted hemodialysis access port 106. Access needle assembly 50 includes a tubular needle 144, an obturator 146, an interface portion 148, a conduit portion 150, and a depth block 152.

Tubular needle 144 has an internal cavity 154 including an open leading end 156 having a beveled edge 158 and an open trailing end 160 opposite the leading end. Trailing end 160 includes a removable cap 162 for closing the trailing end. Cavity 154 includes a side outflow opening 164 adjacent trailing end 160. Side outflow opening 164 is typically positioned on the same side of tubular needle 144 as beveled edge 158.

Obturator 146 is configured to be removably positioned within tubular needle 144. Obturator 146 typically has a cross-section that is substantially equivalent to a cross-section of tubular needle 144 thereby filling cavity 154 when positioned within the tubular needle. Obturator 146 is inserted in tubular needle 144 so that the needle can be inserted through plug 118 without coring and damaging the plug. An example of an obturator used in a hemodialysis is provided in U.S. Pat. No. 6,206,851, which is incorporated by reference as if fully disclosed herein in its entirety.

Interface portion 148 includes a central cavity 166. Tubular needle 144 extends through central cavity 166 so that interface portion 148 is positioned adjacent opening 164, Interface portion 148 includes a flanged portion 168 extending substantially perpendicular from central cavity 166 and positioned concentrically over opening 164. Interface portion 148 includes a lower portion 169 that is configured to removably mate with depth block 152. Flanged portion 168 directs flow through opening 164 in tubular needle 144 when the needle is capped. Interface portion 148 is typically manufactured from a thermoplastic material but can also be manufactured from other materials capable for such uses.

Conduit portion 150 fluidly connects with cavity 166 via flanged portion 168 of interface portion 148. Conduit portion 150 is typically formed from a thermoplastic tubing.

Depth block 152 is joined with interface portion 148 below flanged portion 168. Depth block 152 includes a lower end 170 opposite interface portion 148 that contacts a patient 172 thereby defining a depth D that tubular needle 144 penetrates the patient. In some embodiments, depth block 152 is substantially cylindrical. Typically, tubular needle 144 will be packaged with a ring 174 including a plurality of depth blocks 152. In this way, the particular one of depth blocks 152 selected for use can vary depending on a desired depth of penetration. When mounted on tubular needle 144, depth block 152 limits the depth of penetration by the needle. Typically, at the time port 106 is implanted in a patient and for the first few punctures of the port by tubular needle 144, the exact depth to the middle of artery 108 is determined by the surgeon and other users. Using the determined exact depth, one of depth blocks 152 can be selected so as to have a length that limits the depth of penetration so leading end 156 of tubular needle 144 is positioned in artery 108 when fully inserted.

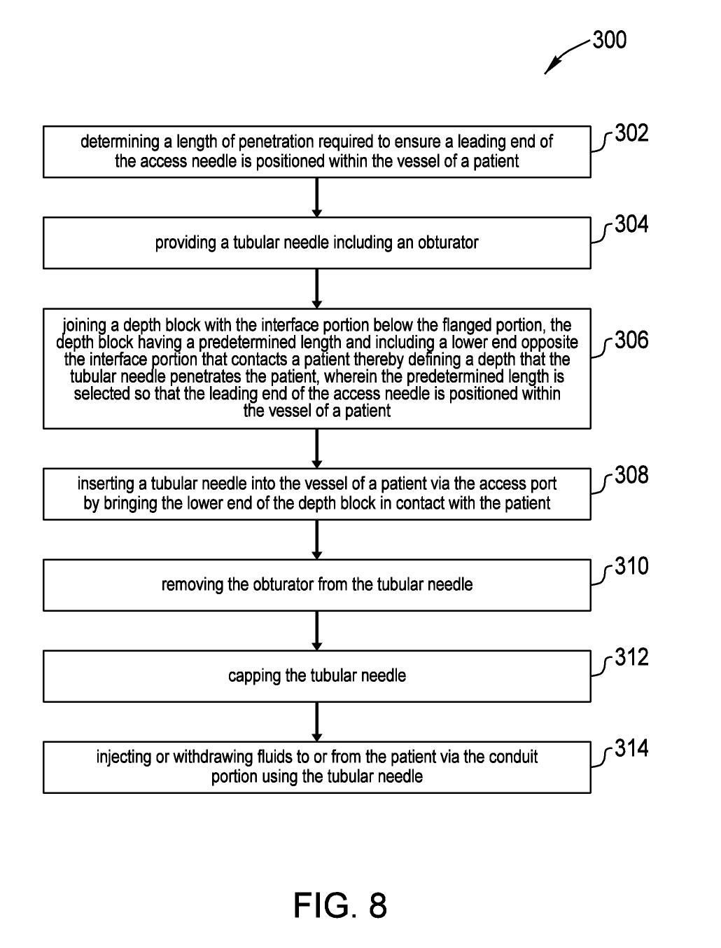

Referring now to FIG. 8, some embodiments of the disclosed subject matter include a method 300 of limiting the depth of penetration of an access needle for use with implanted hemodialysis access ports. At 302, method 300 first includes determining a length of penetration required to ensure a leading end of the access needle is positioned within a vessel of a patient. At 304, a tubular needle such as tubular needle 144 is provided. At 306, a depth block is joined with the interface portion below the flanged portion. The depth block typically has a predetermined length and includes a lower end opposite the interface portion that contacts a patient thereby defining a depth that the tubular needle penetrates the patient. The predetermined length is selected so that the leading end of the access needle is positioned within a vessel of a patient. At 308, the tubular needle, which includes an obturator positioned therein, is inserted into the vessel of the patient via the access port by bringing the lower end of the depth block in contact with the patient. At 310, the obturator is removed from the tubular needle. At 312, the tubular needle is capped. At 314, fluids are injected or withdrawn to or from the patient via the conduit portion using the tubular needle.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. An access needle assembly for use with implanted hemodialysis access ports, said assembly comprising:
a tubular needle having an internal cavity including an open leading end having a beveled edge and an open trailing end opposite said leading end, said trailing end including a removable cap for closing said trailing end, said cavity including a side outflow opening adjacent said trailing end;
an obturator configured to be removably positioned within said tubular needle, said obturator having a cross-section that is substantially equivalent to a cross-section of said tubular needle thereby filling said cavity when positioned within said tubular needle;
an interface portion including a central cavity, said tubular needle extending through said central cavity so that said interface portion is positioned adjacent said opening, said interface including a flanged portion extending substantially perpendicular from said central cavity and positioned concentrically over said opening;
a conduit portion fluidly connected with said cavity via said flanged portion of said interface portion; and
a depth block joined with said interface portion below said flanged portion, said depth block including a lower end opposite said interface portion that contacts a patient thereby defining a depth that said tubular needle penetrates the patient.

2. A device according to claim 1, wherein said opening is on the same side of said needle as said beveled edge.

3. A device according to claim 1, wherein said depth block is substantially cylindrical.

4. A device according to claim 3, wherein said interface portion has a lower portion that mates with said depth block.

5. A device according to claim 1, wherein said depth block is replaceable with others having varying lengths depending on a desired depth of penetration.

6. A system for providing hemodialysis, said system comprising:
an access module including a hemodialysis access port for implanting in a patient, said port including a chimney adapted to be connected to a vessel such that an interior portion of said chimney is in fluid communication with said vessel, a ring adapted to fit securely within said chimney, a collar adapted to fit securely around said chimney, a plug adapted to fit securely within said ring, and a port outer casing partially surrounding the vessel and the collar, said outer port casing including an anti-compression portion adapted to prevent compression of said catheter wherein fluid flowing within the vessel is blocked from passing through said chimney by said plug; and
an access needle module including an access needle assembly for use with an implanted hemodialysis access port, said access needle assembly including
a tubular needle having an internal cavity including an open leading end having a beveled edge and an open trailing end opposite said leading end, said trailing end including a removable cap for closing said trailing end, said cavity including a side outflow opening adjacent said trailing end,
an obturator configured to be removably positioned within said tubular needle, said obturator having a cross-section that is substantially equivalent to a cross-section of said tubular needle thereby filling said cavity when positioned within said tubular needle,
an interface portion including a central cavity, said tubular needle extending through said central cavity so that said interface portion is positioned adjacent said opening, said interface including a flanged portion extending substantially perpendicular from said central cavity and positioned concentrically over said opening,
a conduit portion fluidly connected with said cavity via said flanged portion of said interface portion, and
a depth block joined with said interface portion below said flanged portion, said depth block including a lower end opposite said interface portion that contacts a patient thereby defining a depth that said tubular needle penetrates the patient.

7. A system according to claim 6, wherein said opening is on the same side of said needle as said beveled edge.

8. A system according to claim 6, wherein said depth block is substantially cylindrical.

9. A system according to claim 8, wherein said interface portion has a lower portion that mates with said depth block.

10. A system according to claim 6, wherein said depth block is replaceable with others having varying lengths depending on a desired depth of penetration.

11. A method of limiting the depth of penetration of an access needle for use with implanted hemodialysis access ports, said method comprising:
determining a length of penetration required to ensure a leading end of the access needle is positioned within a vessel of a patient;
providing a tubular needle having an internal cavity including an open leading end having a beveled edge and an open trailing end opposite said leading end, said trailing end including a removable cap for closing said trailing end, said cavity including a side outflow opening adjacent said trailing end, said tubular needle including an obturator configured to be removably positioned within said tubular needle, said obturator having a cross-section that is substantially equivalent to a cross-section of said tubular needle thereby filling said cavity when positioned within said tubular needle, said needle including an interface portion including a central cavity, said tubular needle extending through said central cavity so that said interface portion is positioned adjacent said opening, said interface including a flanged portion extending substantially perpendicular from said central cavity and positioned concentrically over said opening, said tubular needle including a conduit portion fluidly connected with said cavity via said flanged portion of said interface portion;
joining a depth block with said interface portion below said flanged portion, said depth block having a predetermined length and including a lower end opposite said interface portion that contacts a patient thereby defining a depth that said tubular needle penetrates the patient, wherein said predetermined length is selected so that said leading end of said access needle is positioned within a vessel of a patient;
inserting a tubular needle into the vessel of the patient via the access port by bringing said lower end of said depth block in contact with the patient;
removing said obturator from said tubular needle;
capping said tubular needle; and
injecting or withdrawing fluids to or from the patient via said conduit portion using said tubular needle.

12. A method according to claim 11, wherein said opening is on the same side of said needle as said beveled edge.

13. A method according to claim 11, wherein said depth block is substantially cylindrical.

14. A method according to claim 13, wherein said interface portion has a lower portion that mates with said depth block.

15. A method according to claim 11, wherein said depth block is replaceable with others having varying lengths depending on a desired depth of penetration.
